(19) United States
(12) Patent Application Publication
(10) Pub. No.: US 2024/0285962 A1
(43) Pub. Date: Aug. 29, 2024

(54) SYSTEM FOR NEUROSTIMULATION APPLICATIONS

(71) Applicant: INNERVIA BIOELECTRONICS SLU, Barcelona (ES)

(72) Inventors: Jurriaan BAKKER, Heeze (NL); Matteo DONEGA, Huningue (FR); Elisa RODRIGUEZ, Valladolid (ES)

(21) Appl. No.: 18/568,653

(22) PCT Filed: Jun. 10, 2022
(86) PCT No.: PCT/EP2022/065882

§ 371 Date: Dec. 8, 2023

(30) Foreign Application Priority Data
Jun. 11, 2021 (EP) 21382525.0

(51) Int. Cl. A61N 1/40; A61N 1/06

(52) U.S. Cl. CPC A61N 1/40; A61N 1/06

(57) ABSTRACT

The present invention refers to a system for peripheral nerve stimulation, in particular for shaping of the field potentials and/or electric fields and/or second spatial derivatives of the electrical potential parallel and/or non-parallel to the nervous structure, comprising at least one neurostimulation device with at least one stimulation means, preferably a plurality of stimulation means forming a stimulation array, and a control unit. The control unit is configured to provide a nerve stimulus, in particular a nerve stimulus sequence comprising multiple subsequent nerve stimulus pulses, to a nervous structure via the neurostimulation device, wherein the control unit is further configured to provide and apply a conditioning sequence with a least one pulse in advance and/or subsequent to the application of the nerve stimulus via the neurostimulation device to the nervous structure. The conditioning sequence is configured such that an onset response and/or an offset response and/or a neural response overshoot by the neural structure can be limited.

The present invention relates to a system for peripheral nerve stimulation (PNS), in particular for shaping of the field potentials and/or electric fields and/or second spatial derivatives of the electrical potential parallel and/or non-parallel to the nervous/nerve structure/fibers.

In this relation, neuroprosthetic devices are powerful tools to monitor, prevent and treat neural diseases, disorders and conditions by interfacing electrically with the nervous system. They are capable of recording and stimulating electrically neural activity once implanted in the nervous tissue. Currently, most neuroprosthetic technologies apply electrodes interfacing with neural tissue.

Though high frequency alternating current (HFAC) can block nerve conduction, the block is invariably preceded by an onset response, and/or the block is followed by an offset response when the HFAC application is turned off again, which is/are a period of repetitive nerve firing. HFACs can produce nerve conduction block.

Axons can become blocked within milliseconds after the application of the HFAC, remain blocked for as long as the HFAC persists and revert within seconds to their normal state of activity when the HFAC is discontinued. These features make the HFAC block extremely appealing for potential clinical uses.

However, one significant drawback of the HFAC block for clinical applications is the brief but intense volley of activity produced in the nerve each time the HFAC is turned on or off. This volley of activity is referred to as the so called “onset response”/“offset response”, depending on the activation/deactivation of the HFAC.

Conventional methods have tried to apply amplitude ramping in order to overcome the onset, without success, e.g. as addressed by J D Miles et al 2007 J. Neural Eng. 4 390.

Other methods applied amplitude ramps from non-zero amplitudes, which is unsuccessful in reducing initial onsets, but has some effect in reducing subsequent onsets Vrabec2019.

Yi2020 have tried simulations of axon models in combination with particle swarm optimization (PSO) to find stimulation waveforms that successfully reduce onsets. This study is limited to simulations only and does not provide easy to apply waveforms for clinical applications, e.g., it is not directly translatable to electronics, and does not by default meet safe (charge balanced) stimulation limits.

Ackerman2009 suggests that a bipolar electrode with a separation distance of 1.0 mm minimizes current delivery while producing high frequency block with a minimal onset response in the rat sciatic nerve. The results presented in this study demonstrate that the bipolar electrode contact SD affects both the amount of current required to achieve complete neural conduction block and the size of the onset response. The trends in these responses however do not move in the same directions. This suggests that different mechanisms may be responsible for the observed phenomena.

It is an object of the invention to provide a system, in particular for neurostimulation applications, which is capable of providing an enhanced nerve blocking/modulation, preferably a high frequency nerve modulation and/or a low frequency nerve modulation, by specific conditioning of the nerve/nervous structure/fiber in order to limit onset responses and/or offset responses and/or neural response overshoots.

The aforementioned object is solved by the subject-matter of the independent claim 1. Advantageous configurations of the invention are described in dependent claims.

According to the present invention a system for peripheral nerve stimulation, in particular for shaping of the field potentials and/or electric fields and/or second spatial derivatives of the electrical potential parallel and/or non-parallel to the nervous structure, which comprises at least one neurostimulation device with at least one stimulation means, preferably a plurality of stimulation means forming a stimulation array, and a control unit, wherein the control unit is configured to provide a nerve stimulus, in particular a nerve stimulus sequence comprising multiple subsequent nerve stimulus pulses, to a nervous/nerve structure/fiber via the neurostimulation device. The control unit is further configured to provide and apply a conditioning sequence with at least one pulse in advance and/or subsequent to the application of the nerve stimulus via the neurostimulation device to the nervous/nerve structure/fiber, wherein the conditioning sequence is configured such that an onset response and/or an offset response and/or a neural response overshoot by the neural/nerve structure/fiber can be limited/reduced.

The present invention is based on the idea to provide a pre-/post-conditioning of the nervous structure in order to achieve a smoother reaction/response to a change of electrical field/electrical charge in the nervous structure.

For a high frequency stimulus, in particular an HFAC to induce a nerve block, the onset response and/or the offset response by the nervous tissue may be reduced/limited. Moreover, the overshoot response of the tissue in the course of low frequency stimuli may be reduced/limited.

Further, by a specific shaping of the field potentials and/or electric fields and/or second spatial derivatives of the electrical potential parallel and/or non-parallel to the nervous/nerve structure/fibers the responses of the nervous/nerve structure/fibers may also be reduced/limited. Such specifically shaped fields within and/or around the nervous structure may be achieved according to the present invention by an appropriate design of the neurostimulation device and/or a specific control and/or regulation of the application of nerve stimulus along the extension of the neurostimulation device.

In the sense of the present invention the term nerve modulation may be understood to refer to a modulation of the nerve response, either by activation/modulation, blocking or a combination thereof.

In particular, the at least one stimulation means can be provided as an electrode or as a coil. For example, the neurostimulation device can be provided in form of an electrode device, in particular as transcutaneous electrical nerve stimulation (TENS) electrode(s), spinal cord stimulation electrode(s) such as paddle lead(s) or percutaneous lead(s), and/or in form of a nerve cuff for peripheral nerve stimulation.

In in all embodiments of the disclosure, the electrode device can have at least one electrode comprising graphene, in particular being made of graphene or a graphene-based material or comprising a graphene (based) coating.

Preferably, different forms of graphene (based) materials may be used in the context of the present invention, like e.g. reduced graphene oxide (rGO), graphene oxide, chemical vapour deposited graphene (CVD Graphene) or any other potential form of graphene.

In particular, such graphene based materials can provide improved electrical and mechanical properties, e.g. a beneficial flexibility of the resulting electrode.

Such graphene electrodes particularly provide higher safe charge injection capacity as well as the signal-to-noise ratio/performance can be improved. Thereby, the electrode size can be reduced, even if the same amount of electrodes is maintained.

Hence, along a cross-section of the electrode device the cross-sectional area of the at least one electrode can be reduced.

Moreover, such graphene-based electrodes can provide for a safe electrical interface in aqueous environments, like in the context of neuromodulation of nervous tissue.

The control unit can be provided, for example, as an application-specific integrated circuit (ASIC), a field programmable gate array (FPGA) and/or in form of a microprocessor comprising pre-programmed waveform sequences.

The control unit may be integrated, at least partially integrated, in the neurostimulation device and/or may be provided, at least partially provided, as an external control unit.

The neurostimulation device can comprise one stimulation means or a plurality of stimulation means. A nerve stimulus can be applied via one stimulation means, multiple of the plurality of stimulation means or via all of the plurality of stimulations means. In particular, during a nerve stimulus sequence nerve stimulus pulses can be provided via one or multiple of the stimulations means in subsequent or in parallel manner. Thus, a variable pattern of nerve stimulus pulses being provided can be defined.

The nerve stimulus can be provided by the control unit via the stimulation means in form of e.g. an HFAC or in form of a low frequency stimulation pulses. In particular, the nerve stimulus may be provided as a sequence of multiple, subsequently applied nerve stimulus pulses being applied to the nerve structure along the extension of the neurostimulation device, i.e. of the plurality of stimulation means preferably forming a stimulation array.

In the context of the present invention a sequence can be considered as at least a single pulse being provided to the nerve structure or as multiple pulses being applied subsequently, e.g. via a single stimulation means, or even in parallel via e.g. multiple stimulation means along the neurostimulation device.

Moreover, a sequence, in particular multiple pulses being subsequently provided/applied, may also comprise various kinds of pulses. In particular, such subsequent pulses of a sequence can deviate from each other in various pulse parameters, e.g. the pulse width, the intensity of the pulse, a frequency, a sinusoidal, monophasic or biphasic shape, inter pulse intervals, i.e. time delays between cathodic and anodic phases, or the like.

For example, in case of a sinusoidal or biphasic pulse such pulse parameters may even vary with respect to the anodic and/or cathodic phase of the respective pulse(s).

Hence, in the sense of the present invention a sequence, e.g. a nerve stimulus sequence or a conditioning sequence, can comprises a combination of highly variable pulses. Alternatively, a sequence can comprise multiple identical, or at least comparable, pulses be provided in subsequent manner.

The conditioning sequence can be provided as a single pulse for preparation/conditioning of the nerve structure. Moreover, the conditioning sequence can be provided in form of multiple pulses being applied in form of a specific sequence via the neurostimulation device.

Hence, it is also possible to apply a specific pattern of pulses via a plurality of stimulation means to the nerve structure as a conditioning sequence. In accordance with the present invention, the pulses of a conditioning sequence can vary with regard to various pulse parameters, as mentioned above.

In consequence, by the conditioning sequence, in particular a specifically designed conditioning sequence of pulses being provided to the nerve structure via the neurostimulation device, the onset/offset/overshoot response(s) of the nervous/nerve structure/fibers can be limited/reduced.

According to one preferred embodiment, the control unit is configured to provide the conditioning sequence comprising at least one of the following:
at least one pre-pulse, preferably a pre-pulse sequence comprising a combination of multiple pre-pulses having same time delays or different time delays than the nerve stimulus;
a frequency ramp;
a frequency ramp in combination with an amplitude ramp;
a frequency ramp in combination with the least one pre-pulse, preferably in combination with a pre-pulse sequence;
at least one pre-pulse, preferably a pre-pulse sequence, in combination with an amplitude ramp;
a combination with the at least one pre-pulse, preferably a pre-pulse sequence, an amplitude ramp and a frequency ramp.

Various options on how to appropriately condition/prepare the nervous structure are available.

For example, a sequence of pre-pulses can be provided which comprise a time delay between each pre-pulse, and/or between its cathodic and anodic phase, which may be the same as the time delay between nerve stimulus pulses of the correspondingly applied HFAC.

A frequency ramp may describe a function by which the amplitude of the nerve stimulus pulses remain the same during the ramping function, whereby the frequency is increased/decreased over time.

In contrast thereto, an amplitude ramp describes a ramp function by which the frequency remains the same during the conditioning sequence whereby the amplitude of the nerve stimulus pulses is increased/decreased over time.

A combination of an amplitude ramp and a frequency ramp may allow for parallel increase of the amplitude as well as of the frequency of the conditioning sequence.

Moreover, a combination of such amplitude/frequency ramps with at least one pre-pulse, or even a pre-pulse sequence, may be provided in terms of a ramp function being interrupted/supplemented at some point(s) in time by additional application of the pre-pulse(s).

For example, a high frequency or a low frequency (i.e. 1-100 Hz) sinusoidal ramp may be applied. In particular, during the low frequency ramp the frequency may be increased up to 20-40 kHz in order to prepare/condition the nervous structure for the application of a subsequent HFAC. In case a conditioning sequence shall be applied after the application of the HFAC, the frequency could be decreased from 20-40 kHz to 1-100 Hz in such sinusoidally formed frequency ramp again.

According to one preferred embodiment, the conditioning sequence is configured to shape the field potentials and/or electric fields and/or second spatial derivatives of the electrical potential parallel and/or non-parallel to the nervous structure (“shaping”), wherein the shaping by the conditioning sequence can be individualized for different spatial areas along the extension of the nervous structure and/or wherein the shaping by the conditioning sequence is provided on a time dependent basis, preferably in a first spatial direction in a first step and in a second spatial direction in a second step.

In particular, the field potentials and/or electric fields and/or second spatial derivatives of the electrical potential parallel and/or non-parallel to the nervous/nerve structure/fiber can be specifically shaped, preferably to reduce/limit onset/offset response and/or an overshoot response by the nervous structure.

Such specific/individualized shaping of the field potentials and/or electric fields and/or second spatial derivatives of the electrical potential parallel and/or non-parallel to the nervous/nerve structure/fiber may be provided with respect to an area/multiple areas along the nervous structure.

Hence, a shaping may be modified/individualized for specific regions/areas along an extension of the nervous structure, by specific control/regulation and/or design of the neurostimulation device at such specific regions/areas.

This can be considered, in the context of the present invention, as an area dependent shaping.

Alternatively or in addition thereto, a shaping may be provided with respect to a time dependent modification of the field potentials and/or electric fields and/or second spatial derivatives of the electrical potential parallel and/or non-parallel to the nervous structure.

Such time dependent shaping may be provided e.g. in two or multiple steps. In particular, the stepwise shaping can cause the field potentials and/or electric fields and/or second spatial derivatives of the electrical potential parallel and/or non-parallel to the nervous structure to change directions during the single steps of shaping.

For example, the field potentials and/or electric fields and/or second spatial derivatives of the electrical potential parallel and/or non-parallel to the nervous structure may be shaped
in a first step to be oriented perpendicular to the nervous structure, as a first direction, and
in a second step to be turned into a second direction parallel to the nervous structure.

In this way, a nerve block could be achieved during/by the first step or subsequent steps. Moreover, onset/offset responses and/or overshoot responses by the nervous structure may be limited/reduced by such time dependent shaping approach in multiple steps.

In another embodiment of the present invention the conditioning sequence can comprise at least one variable parameter, preferably to form a ramp function, wherein further stimulation parameters of the conditioning sequence are at least comparable, preferably similar, to the nerve stimulus (sequence).

Thus, the system, in particular the control unit, provides for a variety of options on how to appropriately condition/prepare the nervous structure in advance to and/or subsequent to the nerve stimulus (sequence/pulse(s)).

In particular, the conditioning sequence can focus on at least one variable parameter, like the frequency, the amplitude, the pulse duration, the inter pulse interval or the like, in order to achieve a (pre-)conditioning of the nervous structure in a stepwise manner.

According to another embodiment the conditioning sequence, preferably referring to a frequency and/or amplitude ramp function, is configured to comprise similar waveform as the nerve stimulus or to deviate from the nerve stimulus with respect to being applied with
a sinusoidal, monophasic or biphasic waveform,
different pulse width(s),
different amplitude(s), and/or
different inter pulse interval(s).

In particular, providing different inter pulse interval(s) may refer to a deviating delay between cathodic and anodic phases of the respective pulses.

Thus, the conditioning sequence can be provided in comparable manner to the corresponding nerve stimulus (sequence) or can deviate therefrom, in order to provide an appropriate preparation/conditioning of the nervous structure before/after the nerve stimulus (sequence).

In a further embodiment, the nerve stimulus sequence is
a high frequency alternating current being provided to the nervous structure in order to achieve high frequency block of the nervous structure or
a low frequency stimulus being provided to the nervous structure, in particular for nerve modulation.

Preferably, the system according to the present invention is configured in such a way to be suitable for low frequency nerve stimulus applications as well as for high frequency applications, like nerve blocking by application of HFAC.

In particular, a response of the nervous structure to the application of nerve stimuli, like onset/offset response or overshoot response, can be shaped/influenced in order to be reduced/limited finally.

In another preferred embodiment the neurostimulation device comprises the at least one stimulation means being configurable such that the nerve stimulus provided via the at least one stimulation means is adaptable along the nervous structure. According to one embodiment the at least one stimulation means is adaptable by modification of the design, in particular of the shape, of the neurostimulation device, the configuration of the stimulation array, and/or the like.

In particular, field potentials and/or electric fields and/or second spatial derivatives of the electrical potential parallel and/or non-parallel to the nervous/nerve structure/fibers may have an impact on the onset modulation function. Hence, changing those with regard to the nervous structure of interest, e.g. by making those less steep (i.e. smoother), can reduce the onset/offset response.

Because the nervous/nerve structures/fibers in a nerve are not homogeneous, but for example grouped in fascicles, and with different motor/sensory afferent/efferent functions, those can be targeted with a dedicated electrode array and/or a (specific) field shaping to reduce the onset/offset response.

Thus, by the system according to the present invention the neurostimulation device, in particular the arrangement and/or the use of the single stimulation means, can be individually adapted in order to form the field potentials and/or electric fields and/or second spatial derivatives of the electrical potential within and/or around the neural structure in appropriate manner.

In particular, the resulting field potentials and/or electric fields and/or second spatial derivatives of the electrical potential can be shaped to achieve a limitation/reduction of the onset/offset response or an overshoot response of the neural structure not only a one specific point but preferably along a certain area/extension of the neurostimulation device.

Such shaping of field potentials and/or electric fields and/or second spatial derivatives of the electrical potential may particularly be dependent on the timing/time dependent and/or area dependent.

For example, in case of a neurostimulation device comprising only a single stimulation means such stimulation means can comprise an individually shape geometrical extension along the neurostimulation device.

In case of a neurostimulation device with a plurality of stimulation means, such stimulation means can form a specific geometric pattern representing a stimulation array which may be adapted and or specifically used, preferably in subsequent, in parallel or any other arbitrary combined manner for appropriately transmission of nerve stimulus pulses.

Furthermore, the (geometrical) shape of the neurostimulation device may be adapted, e.g. its length, its diameter, its outer circumferential geometry, or the like.

Moreover, the control unit can forward appropriate stimulation signals to the single stimulations means of the neurostimulation device, preferably individual stimulation signals, to provide a specific and targeted stimulation of the nervous structure.

In a further embodiment of the present invention the control unit is configured to provide individual stimulation signals to each of the plurality of stimulation means such that the nerve stimulus as provided to the nervous tissue via the plurality of stimulation means can be shaped along the extension of the neurostimulation device. According to another embodiment the control unit is configured to shape the nerve stimulus along the extension of the neurostimulation device by applying weighing factors on the stimulation signal in order to provide individual stimulation signals to each of the plurality of stimulation means.

In the sense of the present invention the control unit provides stimulation signals to the stimulation means in order to apply/transmit a nerve stimulus (sequence) to the nervous/nerve structure/fiber.

Such stimulation signals may be adapted by the control unit, e.g. by the application of weighing factors for amplification of the stimulation signal, for specific stimulation means of the neurostimulation device. Preferably, the stimulation means can be individually controlled and/or regulated such that by individual stimulation signals the nerve stimulus of the respective stimulation means can be individualized.

A weighing factor may be “0” (value: zero) in order to “switch off” a single stimulation means or smaller than 1.0 (value “one”) in order to decrease correspondingly transmitted nerve stimulus pulse(s). In case the nerve stimulus pulse(s) shall be applied without any amplification, the weighing factor can be 1.0. If an amplified nerve stimulus (pulse/sequence) shall be transmitted via a specific stimulation means, the corresponding stimulation signal can be amplified, in particular increased, by a weighing factor of e.g. 1.1, 1.5, 2 or the like.

Hence, the application of a nerve stimulus (sequence) via the plurality of stimulation means may be adapted and shaped by the control unit, in particular by the application of weighing factors to the stimulation signals respectively, such that a specific and targeted application of nerve stimulus pulses is achievable.

Thus, a stimulation signal for one of the plurality of stimulation means can even be reduced to zero or at least close to zero, while other stimulation means are provided with an amplified stimulation signal, by multiplication with a weighing factor higher than the 1.0.

Moreover, multiple of the plurality of stimulation means can receive the same stimulation signal for providing the nerve stimulus to the nervous tissue while other stimulation means of the plurality receives adapted stimulation signals. Hence, each stimulation means can receive a specific stimulation signal, provided by the control unit.

Alternatively, the control unit can provide the same stimulation signal to all of the plurality of stimulation means whereby the structure of the stimulation means, in particular the structural implementation of the array of stimulation means, is configured to provide individualized nerve stimulus by the respective stimulation means.

In a further preferred embodiment the neurostimulation device is an implantable pulse generator a transcutaneous stimulator device, e.g. a skin patch, or the like.

Consequently, the system according to the present invention may be specifically designed/configured to be used in PNS applications.

In particular, the nerve blocking mechanism by applying HFAC or the smooth initiation of a low frequency nerve stimulus sequence can be particularly utilized in the context of PNS.

Hence, a specific and individual stimulation of the nervous/never structure(s)/fiber(s) can be achieved, e.g. as the application of HFAC for initiating nerve block allows for targeted stimulation/non-stimulation of the nervous tissue.

Further details and advantages of the present invention shall now be disclosed in connection with the enclosed drawings.

It is shown in:

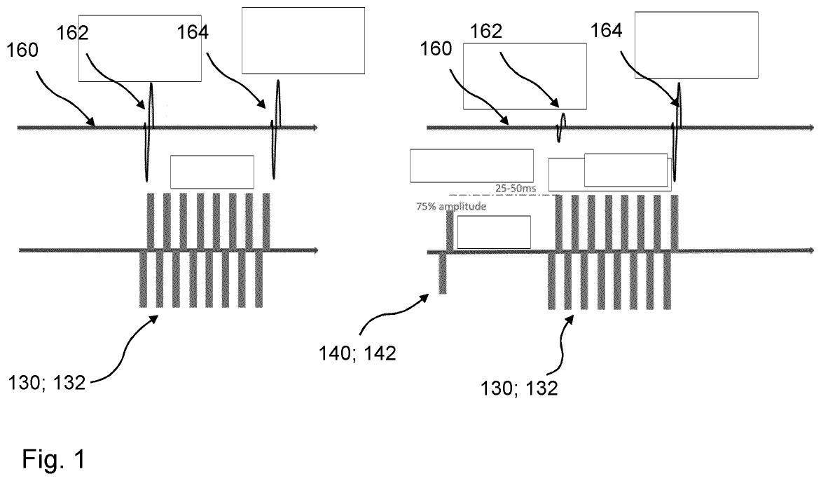

FIG. 1 a schematic illustration of an onset/offset response due to immediate application of a nerve block stimulus according to the prior art in comparison to the application of a (pre-)conditioning sequence according to one embodiment of the system;

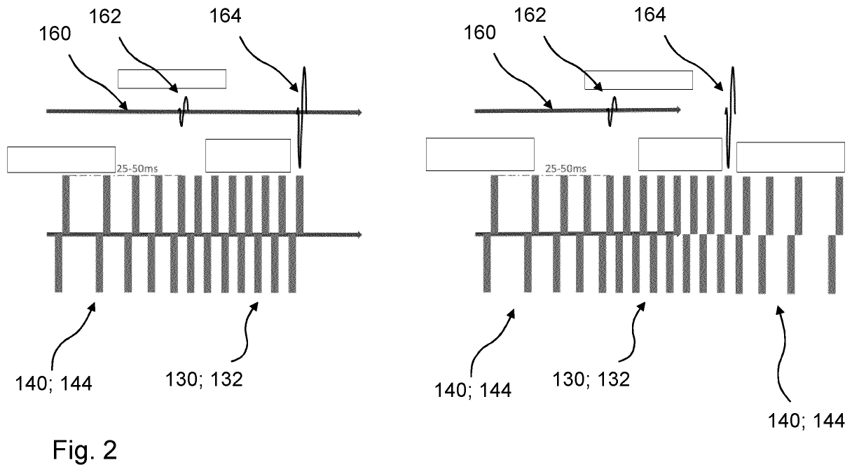

FIG. 2 a schematic illustration of the application of a conditioning sequence according to another embodiment of the system;

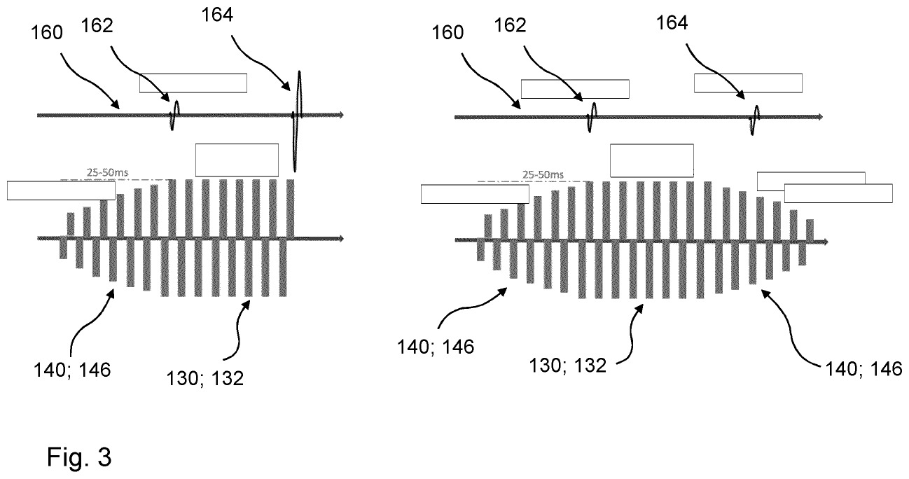

FIG. 3 a schematic illustration of the application of a conditioning sequence according to another embodiment of the system;

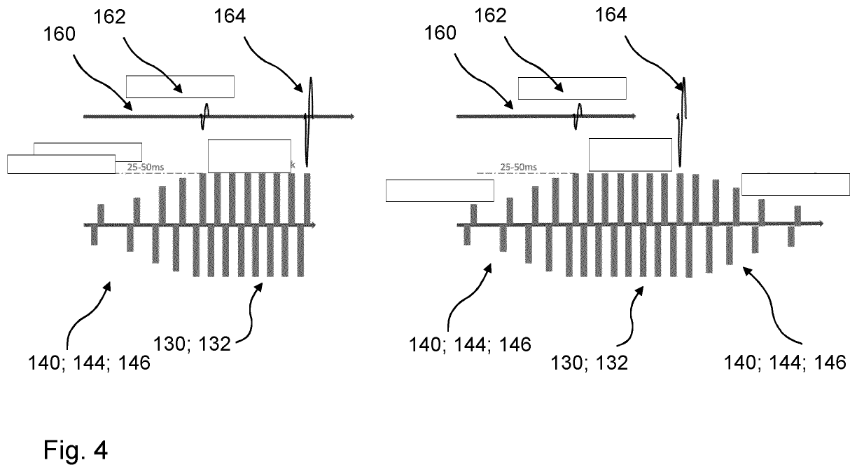

FIG. 4 a schematic illustration of the application of a conditioning sequence according to another embodiment of the system;

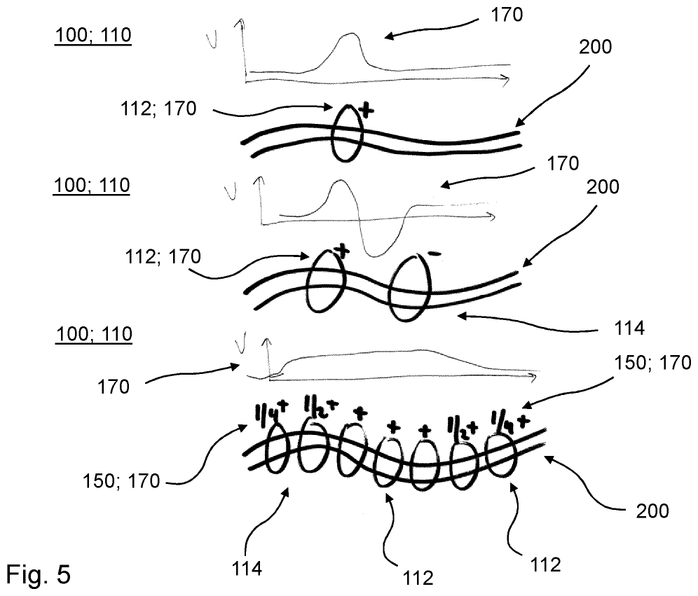

FIG. 5 a comparison of neurostimulation device configurations according to an embodiment of the system; and

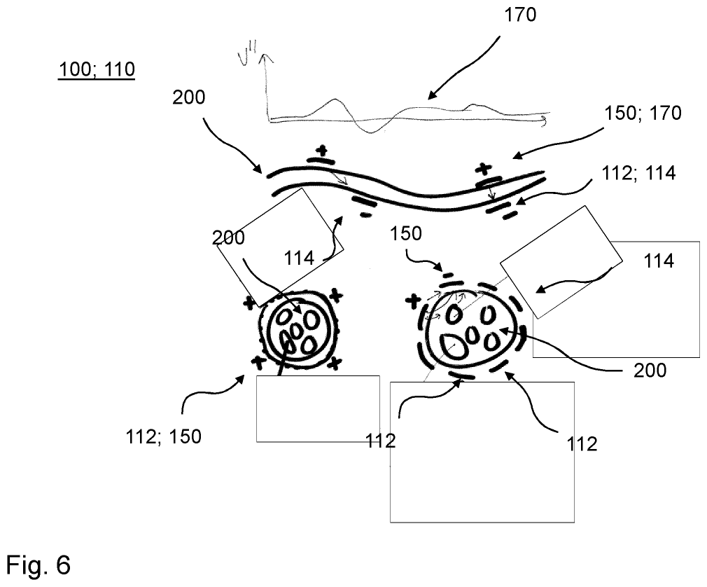

FIG. 6 a comparison of neurostimulation device configurations according to another embodiment of the system.

In FIG. 1 a schematic illustration of an onset/offset response 162; 164 of a stimulated nervous/nerve structure/fiber 200 due to (immediate) application of a nerve block stimulus 130 according to the prior art is shown, in comparison to the application of a (pre-) conditioning sequence 140 according to one embodiment of the system 100.

According to FIG. 1 a nerve stimulus sequence 130 with multiple nerve stimulus pulses 132, in form of high frequency alternating current for inducing a nerve block, is applied.

In particular, the nerve stimulus sequence 130 is applied in form of biphasic pulses 132.

As can be gathered from the left diagram of FIG. 1, illustrating the application of an HFAC according to the prior art, an onset/offset response 162; 164 by the stimulated nerve structure 200 is caused at the start/end of the HFAC application, as can be gathered from the corresponding response of the nervous structure 160.

By simple and immediate application of an HFAC as nerve stimulus sequence 130, an uncontrolled, intense volley of activity within the nervous structure 200 is caused/induced.

In contrast thereto, the right illustration in FIG. 1 shows the application of a nerve stimulus sequence 130, in form of an HFAC, in combination with a single pre-pulse 142 in advance to the nerve stimulus sequence 130.

According to FIG. 1, the pre-pulse 142 is applied with an amplitude of 75% of the intensity of the subsequent HFAC, namely the nerve stimulus sequence 130.

The single pre-pulse 142 is applied between 1 us to 1 min, preferably between 1 us to 1 s, 1 us to 100 ms and even more preferably between 25 to 50 ms before the start of the nerve stimulus sequence 130.

The pre-pulse 142 is applied as a biphasic pulse sequence.

As can be gathered from a comparison of the left and right illustrations of FIG. 1, the application of the pre-pulse 142, in advance to the nerve stimulus sequence 130 cause a limitation/reduction of the onset response 162 in the response (signal) of the nervous structure 200.

This can particularly be gathered from FIG. 1 (right illustration) as only the onset response 162 is limited/reduced, wherein the subsequent offset response 164 is not reduced/limited on the immediate end of the nerve stimulus sequence 130.

In FIG. 2 to FIG. 4 schematic illustrations of the application of a conditioning sequence according to further embodiments of the system 100 are shown.

According to FIG. 2 a frequency ramp 144 is applied as a conditioning sequence 140.

The frequency ramp 144 is applied as a conditioning sequence 140 with a plurality of pulses having the same intensity/amplitude as the subsequent nerve stimulus sequence 130, preferably an HFAC.

The conditioning sequence 140 comprising a frequency ramp 144 is applied over a time of 25 to 50 ms before the nerve stimulus sequence 130 starts.

In the context of the frequency ramp 144 as conditioning sequence 140, the frequency of pulses being applied is increased over time, preferably up to a frequency which matches the frequency of the subsequent nerve stimulus sequence 130.

Moreover, in the right illustration of FIG. 2, also a conditioning sequence 140 in form of a frequency ramp 144 is applied subsequent to the end of the nerve stimulus sequence 130, causing a frequency to be reduced over time, e.g. over another 25-50 ms.

As can be seen from the corresponding responses of the nervous structure 160, the conditioning sequence 140 before the nerve stimulus sequence 130 causes the onset response 162 along the response of the nervous structure 160 to be limited/reduced.

Moreover, if a further (post-)conditioning sequence 140 is applied after the end of the nervous stimulus sequence 130 (right illustration of FIG. 2), also the offset response 164 can be reduced/limited, as indicated along the response of the nervous structure 160.

In FIG. 3 the conditioning sequence 140 is provided in form of an amplitude ramp 146.

The amplitude of the pulses being applied to form the conditioning sequence 140 is increased over time, preferably until an amplitude of the subsequent nervous stimulus sequence 130 is provided.

The amplitude ramp 146 can be provided with a constant frequency, preferably the frequency of the subsequent nerve stimulus sequence 130.

According to FIG. 3, the conditioning sequence 140 is provided over a time range of 25 to 50 ms in advance to the nerve stimulus sequence 130.

Again, in the right illustration of FIG. 3 a further conditioning sequence 140 is applied after the end of the nerve stimulus sequence 130, in particular in form of an amplitude ramp 146 comprising a decrease of the amplitude/intensity over time.

As can be seen from the corresponding response of the nervous structure 160, the onset response 162 can be limited/reduced by the (pre-)conditioning sequence 140; 146.

Moreover, also the (post-)conditioning sequence 140; 146, being applied after the end of the nerve stimulus sequence 130, can reduce/limit the offset response 164 along the response of the nervous structure 160.

The conditioning sequence 140 in form of an amplitude ramp 146 may be able to reduce onset and offset responses 162; 164 in order to smoothen the overall application of nerve stimulus sequence 130, like an HFAC application.

In FIG. 4, a combination of an amplitude ramp 146 and a frequency ramp 144 is applied as a conditioning sequence 140 before and/or after the nerve stimulus sequence 130.

In particular, before the nerve stimulus sequence 130, the amplitude as well as the frequency is increased over a time of 25 to 50 ms during the conditioning sequence 140; 144; 146, preferably up to a frequency and an amplitude of the subsequent nerve stimulus sequence 130.

As can be seen from the respective responses of the nervous structure 160, the onset response 162 is limited for both embodiments as illustrated in FIG. 4. Moreover, also the offset response 164 can be limited in case a corresponding conditioning sequence 140 is applied after the nerve stimulus sequence 130.

In FIG. 5 and FIG. 6 a comparison of neurostimulation device 110 configurations according to various embodiments of the system 100 are shown.

FIG. 5 illustrates three different embodiments (top, middle, bottom) of configurations of a neurostimulation device 110 of a system 100.

The three embodiments differ from each other with respect to their amount of stimulations means 112, which can form a stimulation array 114.

According to FIG. 5 the stimulation means 112 are arranged along a nervous structure 200.

In particular, FIG. 5 can schematically illustrate the arrangement of nerve cuffs, e.g. for PNS.

Preferably, the stimulations means 112 in FIG. 5 may be provided in form of coils.

Alternatively, the stimulations means 112 can be provided in form of electrodes, e.g. for a (rod-like) nerve stimulation electrode.

The single stimulation means 112 can be provided with different polarities (see middle and top illustrations of FIG. 5) in order to shape the field potentials and/or electric fields and/or second spatial derivatives of the electrical potential parallel and/or non-parallel to the nervous structure 170 along the nervous structure 200.

Moreover, weighing factors 150 may be applied for the shaping of the field potentials and/or electric fields and/or second spatial derivatives of the electrical potential parallel and/or non-parallel to the nervous structure 170, preferably applied by a control unit on stimulation signals which are forwarded to the respective stimulation means 112 to transmit nerve stimulus pulses 132 to the nervous structure 200.

For example, by the application of weighing factors 150 an area dependent shaping can be provided.

By the design of the neurostimulation device 110, in particular of the stimulation means 112 forming a specific stimulation array 114, and/or by the control unit providing specifically adapted stimulation signals, e.g. by the application of weighing factors 150, the electrical field (lines) can be shaped and adapted to the nervous structure 200.

This can particularly be gathered from the respective curves of electrical field potentials 170 for the different embodiments according to FIG. 5.

The top embodiment of FIG. 5 comprises a single stimulation means 112 with a positive potential, thus causing an unipolar, anodic phase of field potential.

The middle embodiment of FIG. 5 comprises two stimulations means 112 having different polarities, thus causing a bipolar field potential with an anodic phase and a cathodic phase.

The bottom embodiment of FIG. 5 comprises a plurality of stimulation means 112 having a positive polarity with different weighing factors 150, thus causing a rather constant anodic phase along the extension of the nervous structure 200.

Alternatively, a time-wise application of weighing factors 150 may provide a time dependent shaping.

As a further alternative, by causing/providing a stepwise (re-)orientation of the field potentials and/or electric fields and/or second spatial derivatives of the electrical potential parallel and/or non-parallel to the nervous structure, preferably in different directions subsequently, a time dependent shaping may be achieved.

In FIG. 6, cross sections of an embodiment referring to a nerve cuff (bottom left illustration in FIG. 6) and an electrode arrangement (bottom right illustration in FIG. 6), like for a nerve neurostimulation device 110, are shown.

Moreover, FIG. 6 also shows a distribution of the neurostimulation devices 110 along the nervous structure 200 (middle illustration of FIG. 6) as well as corresponding second spatial derivative of the potentials parallel and/or non-parallel to the nervous structure 200 (top illustration of FIG. 6).

The neurostimulation devices 110 in FIG. 6 surround multiple different nerve fibers 200

The embodiment of a nerve cuff as neurostimulation device 110 (left illustration in FIG. 6) shows a stimulation means 112 in form of a coil.

The neurostimulation means 112 has/have the same positive potential around its circumference.

The embodiment of e.g. a nerve electrode as a neurostimulation device 110 comprises multiple electrodes as stimulation means 112 being distributed all around the circumference of the neurostimulation device 110.

The stimulation means 112 form a stimulation array 114.

The polarity of the single stimulation means 112 can be controlled/alternated such that different potentials can be applied around the circumference of the neurostimulation device 110.

According to FIG. 6, the weighing factors 150 being applied for the respective stimulations means may be 1.

As can be further seen from the second spatial derivative potential illustration of FIG. 6 (illustration at the top), the two embodiments cause different potentials to occur along the nervous structure 200.

The embodiment referring to a nerve cuff having multiple coils as stimulation means 112 (left illustration of FIG. 6) cause a biphasic potential along the nervous structure 200, wherein the electrode configuration of a neurostimulation device 110, like a nerve stimulation electrode, (right illustration of FIG. 6) causes an uniphasic potential along the nervous structure 200.

Thus, the shape and configuration of the neurostimulation device 110 as well as individual shaping of stimulation signals by the control unit, e.g. by using weighing factors 150, allow for an individualization of the electrical potential along the nervous structure 200, in an area dependent and/or time dependent manner, in order to achieve a limitation/reduction of onset/offset responses, e.g. in the context of HFAC applications to provide nerve block.

In summary, the system 100 according to the present invention can provide a highly variability for high or low frequency nerve stimulus (sequence) 130 applications, whereby uncontrolled nerve response in terms of onset response 162 and/or offset response 164 and/or overshoot responses can be limited/reduced.

In particular, a conditioning sequence 140 may be applied before and/or after a nerve stimulus (sequence) 130, like e.g. an HFAC for achieving nerve block, in order to reduce/limit the response of the nervous structure 160; 200.

Such conditioning sequences 140 can be provided in different manner, e.g. in form of a pre-pulse (sequence) 142, a frequency ramp 144 or an amplitude ramp 146. Further, combinations of such measures are providable as well.

Moreover, the onset/offset responses 162; 164 of the nervous/nerve structure/fiber 200 may also be limitable/reducible by a specific design of the neurostimulation device 110, in particular with respect to the configuration of the stimulation means 112 in form of a stimulation array 114, and/or by the control unit providing stimulation signals being amplified on basis of weighing factor(s)150.

Thus, the present invention also allows for specific shaping of the field potentials and/or electric fields and/or second spatial derivatives of the electrical potential parallel and/or non-parallel to the nervous structure 170 along the extension of the stimulation device 110, having at least one stimulation means 112.

### REFERENCE NUMERALS

100 System
110 Neurostimulation device
112 Stimulation means
114 Stimulation array
130 Nerve stimulus (sequence)
132 Nerve stimulus pulse
140 Conditioning sequence (of pulses)
142 Pre-pulse (sequence)
144 Frequency ramp
146 Amplitude ramp
150 Weighing factor
160 Response of nervous structure
162 Onset response
164 Offset response
170 Field potentials/electric fields/second spatial derivatives of the electrical potential parallel and/or non-parallel to the nervous structure
200 Nervous structure

1. System for peripheral nerve stimulation, in particular for shaping of the field potentials and/or electric fields and/or second spatial derivatives of the electrical potential parallel and/or non-parallel to the nervous structure,
comprising at least one neurostimulation device with at least one stimulation means, and a control unit,
wherein the control unit is configured to provide a nerve stimulus to a nervous structure via the neurostimulation device,
wherein the control unit is further configured to provide and apply a conditioning sequence with a least one pulse in advance and/or subsequent to the application of the nerve stimulus via the neurostimulation device to the nervous structure,
wherein the conditioning sequence is configured such that an onset response and/or an offset response and/or a neural response overshoot by the neural structure can be limited.

2. System according claim 1,
wherein
the control unit is configured to provide the conditioning sequence comprising at least one of the following:
at least one pre-pulse, including a pre-pulse sequence comprising a combination of multiple pre-pulses having same time delays or different time delays than the nerve stimulus;
a frequency ramp;
a frequency ramp in combination with an amplitude ramp;
a frequency ramp in combination with the least one pre-pulse in combination with a pre-pulse sequence;
at least one pre-pulse sequence, in combination with an amplitude ramp;
a combination with the at least one pre-pulse.

3. System according to claim 1,
wherein
the conditioning sequence is configured to shape the field potentials and/or electric fields and/or second spatial derivatives of the electrical potential parallel and/or non-parallel to the nervous structure,
wherein the shaping by the conditioning sequence is individualized for different spatial areas along the extension of the nervous structure and/or
wherein the shaping by the conditioning sequence is provided on a time dependent basis in a first spatial direction in a first step and in a second spatial direction in a second step.

4. System according to claim 1,
wherein
the conditioning sequence can comprise at least one variable parameter to form a ramp function, including a frequency ramp function and/or an amplitude ramp function, wherein further stimulation parameters of the conditioning sequence are at least comparable to the nerve stimulus.

5. System according to claim 1,
wherein
the conditioning sequence, referring to a frequency and/or amplitude ramp function, is configured to comprise similar waveform as the nerve stimulus or to deviate from the nerve stimulus with respect to being applied with
a sinusoidal, monophasic or biphasic waveform,
different pulse width(s),
different amplitude(s), and/or
different inter pulse interval(s).

6. System according to claim 1,
wherein
the nerve stimulus sequence is
a high frequency alternating current being provided to the nervous structure in order to achieve high frequency block of the nervous structure or
a low frequency stimulus being provided to the nervous structure.

7. System according to claim 1,
wherein
the neurostimulation device comprises the at least one stimulation means being configurable such that the nerve stimulus provided via the at least one stimulation means is adaptable along the nervous structure.

8. System according to claim 7,
wherein
the at least one stimulation means is adaptable by modification of the design, in particular of the shape, of the neurostimulation device, the configuration of the stimulation array, and/or the like.

9. System according to claim 1,
wherein
the control unit is configured to provide individual stimulation signals to each of the plurality of stimulation means such that the nerve stimulus as provided to the nervous structure via the plurality of stimulation means can be shaped along the extension of the neurostimulation device.

10. System according to claim 9,
wherein
the control unit is configured to shape the nerve stimulus along the extension of the neurostimulation device by applying weighing factors on the stimulation signal in order to provide individual stimulation signals to each of the plurality of stimulation means.

11. System according to claim 1,
wherein
the neurostimulation device is an implantable pulse generator or a transcutaneous stimulator device.

12. System according to claim 1 wherein a plurality of stimulation means form a stimulation array, and wherein the nerve stimulus comprises a nerve stimulus sequence comprising multiple subsequent nerve stimulus pulses.
